(19) United States
(12) Patent Application Publication
(10) Pub. No.: US 2026/0325818 A1
(43) Pub. Date: Oct. 22, 2026

(54) METHOD AND APPARATUS FOR IMPLANTATION OF A HEART PUMP

(71) Applicant: The Trustees of Indiana University, Bloomington, IN (US)

(72) Inventor: Mark D. Rodefeld, Carmel, IN (US)

(73) Assignee: The Trustees of Indiana University, Bloomington, IN (US)

(21) Appl. No.: 18/876,439

(22) PCT Filed: Jun. 27, 2023
(86) PCT No.: PCT/US2023/026334

§ 371 Date: Dec. 18, 2024

Related U.S. Application Data
(60) Provisional application No. 63/367,051, filed on Jun. 27, 2022.

(51) Int. Cl. A61M 60/135; A61M 60/216; A61M 60/35; A61M 60/416

(52) U.S. Cl. CPC A61M 60/135; A61M 60/216; A61M 60/35; A61M 60/416

(57) ABSTRACT

Various embodiments herein pertain to implantable devices that are connected to the internal anatomy of an animal by one or more separable fluid ducts that are graftable on one end to the anatomy and connected on the other end to an implantable device such as a blood pump.

### FIELD OF THE INVENTION

The present inventions pertain to methods and apparatus for implanting a device in an animal, and more particularly for implantation of a pumping device in a circulatory system.

### SUMMARY OF THE INVENTION

In one embodiment, the present invention pertains to a pump that is implantable within the circulatory system of an animal. The device is connected to the circulatory system by one or more graftable conduits that connect to vascular tissue. These vascular connections can be performed by suturing. However, the conduit is also in fluid communication with the device at inlet and outlet ports that cannot be sutured to the harder surfaces of the device. Instead, each of the conduits includes a rigid or semi-rigid connection that can be mechanically coupled to a complementary connection on the device body. In some embodiments, these connections are circular flanges held together by a clamp.

Still further embodiments of the present invention pertain to an implantable housing having an external shape the same as an implantable blood pump. The housing is useful for implanting separate ducts grafted into the circulatory system. The proper installation of the ducts can be verified with the housing prior to implantation of the pump.

Yet other embodiments of the present invention pertain to the suturing connection of separable fluid ducts into the anatomy of an animal, with subsequent attachment of an implantable pump into the animal, the pump being in fluid communication with the fluid ducts and the animal circulatory system.

Various embodiments of the present invention provide one or more of the following aspects, each provided as a non-limiting example:
Seamless PTFE connector on each graftable conduit
4 grafts sutured to each vessel
   Reinforced or non-reinforced graft
   Length specified by surgeon.
If the graft connection is unsatisfactory, the graft can be replaced without incurring the cost of an entire pump.
The pump body is not in the way of the surgeon during suturing of the graftable conduits.
A “blank” housing can be used to assess spacing of the conduits.
Pump can be inserted after graft patency is assured.
Use quick-connect tool to attach the 4 grafts to the pump
Facilitates surgical implantation, improves accuracy of implantation, reduces risk of adverse events, reduces risk of accidental damage to a high-precision implantable medical device.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions. Further, the figures shown herein may have been created from scaled drawings, scaled models, or from photographs that are scalable. It is understood that such dimensions, or the relative scaling within a figure, are by way of example, and not to be construed as limiting unless so stated in a claim. Persons of ordinary skill will also recognize that CAD renderings may include lines that pertain to changes in the computer model, and not necessarily to component features.

FIG. 1A is a side view of a portion of a circulatory system of an animal with the circulatory system being partly modified in preparation for a Fontan procedure.

FIG. 1B is a view of the circulatory system of FIG. 1A showing the circulatory system as prepared for bypassing operation.

FIG. 1C is a view of the circulatory system of FIG. 1B prior to implantation of a pump in the Fontan junction.

FIG. 1D shows the circulatory system of FIG. 1C with the pump implanted.

FIG. 2A is a close-up of a portion of a circulatory system that is prepared for insertion of a pump.

FIG. 2B shows the circulatory system of FIG. 2A with a pump implanted, the pump being shown in partial cutaway view according to one embodiment of the present invention.

FIG. 3 is an exploded view of an implantable pumping assembly according to another embodiment that includes a central blood pressure-boosting device and four graftable conduits.

FIG. 4 is a cross sectional representation of an implanted pumping assembly connected by four graftable conduits to a circulatory system.

FIG. 5A shows a perspective photographic representation of four conduit blanks prior to their conversion to a graftable configuration.

FIG. 5B shows one of the conduit blanks of FIG. 5A being modified to a graftable configuration.

FIG. 6A shows a perspective photographic representation of a clamp assembly according to one embodiment of the present invention.

FIG. 6B shows a perspective photographic representation of a locking device according to one embodiment of the present invention.

FIG. 6C shows a perspective photographic representation of a clamp assembly according to one embodiment of the present invention.

FIG. 6D shows a method of coupling two flanged attachments together.

FIG. 7A shows a variety of flange configurations useful for alignment of the conduits and housings in some embodiments of the present invention.

FIG. 7B shows another variety of flange configurations useful for alignment of the conduits and housings in some embodiments of the present invention.

### ELEMENT NUMBERING

The following is a list of element numbers used with all of the embodiments, and at least one noun used to describe that element. It is understood that none of the embodiments disclosed herein are limited to these nouns, and these element numbers can further include other words that would be understood by a person of ordinary skill reading and reviewing this disclosure in its entirety.

| | | | |
|---|---|---|---|
| 10 | Circulatory system | 42 | pumping surface |
| 12 | Fontan junction | 44 | outlet, cooling flow |
| 18 | sutures | 50 | Motor |
| 20 | Implantable pumping | 52 | shaft |
| | assembly | 54 | bearings |
| .1 | direction of flow | 60 | graftable conduit |
| 22 | centerline | .1 | conduit, pump inlet |
| 26 | conduit, electrical power | .2 | conduit, pump outlet |
| 30 | housing | .3 | conduit, pump inlet |
| 32 | body | .4 | conduit, pump outlet |
| .1 | wall | 62 | body |
| .2 | interior | .1 | end |
| .3 | exterior | .2 | lumen |
| .4 | chamber | .3 | flexible portion |
| .5 | side | .4 | rigid portion |
| 34 | inlet; fluid connection | 64 | inlet |
| .1 | connection feature | .1 | housing connection end; |
| .2 | sealing face or compression | | means for coupling to |
| | face | | the housing; flange |
| .3 | periphery | .12 | sealing face or compression |
| .4 | bearing support, strut | | face |
| 38 | outlet; fluid connection | .14 | periphery |
| .1 | connection feature | .2 | anatomical connection end; |
| .2 | sealing face or compression | | means for coupling to a |
| | face | | circulatory system |
| .3 | periphery | 68 | outlet |
| .4 | gap | .1 | housing connection end; |
| 40 | pump; impeller | | means for coupling to |
| .12 | sealing face or compression | | the housing; flange |
| | face | 72 | channel |
| .14 | periphery | 74 | lock |
| .2 | anatomical connection end; | 75.1 | threads |
| | means for coupling to a | 75.2 | bolt |
| | circulatory system | 76 | seal |
| 70 | clamp assembly | | |

### Detailed Description of One or More Embodiments

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “various embodiments” or “preferably,” refers to features and aspects that are present in at least one embodiment, but which are optional for some embodiments, it therefore being understood that use of the word “preferably” implies the term “optional.”.

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise explicitly noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

This disclosure relates to methods and apparatus for surgical implantation. When a blood pump is surgically implanted, it is attached to blood vessels or the heart. Surgeons typically suture these attachments to the appropriate anatomic structures, and may then separately attach the pump using a coupling method if the pump is so designed.

In the case of using a pump in a Fontan arrangement of the circulatory system, the space for suturing is critically limited because the pump is placed in situ in an anatomically tight space adjacent to the aorta, trachea, etc. Four blood vessel connections are required in the case of a Fontan procedure, although the present invention also contemplates implantations in “Y” shaped junctions having two inlets and a single outlet, and further contemplates implantations of “Y” shaped junctions having two outlets and a single inlet, and further contemplates any implantable device having at least one fluid connection. Because of the bulk of the pump, it may be difficult to suture each of the connections without being constrained by lack of sufficient space to safely and successfully complete the anastomoses (connections).

Various embodiments of the present invention contemplate a novel method of using pre-formed suturable grafts (by way of example only, expanded polytetrafluorethylene, ePTFE, commonly known as Gore-tex®) to facilitate blood vessel anastomosis. Rigid pre-formed ends of the grafts (PTFE ends, not ePTFE ends) are seamlessly integrated to the flexible portion of the graft and allow for secure connection to the pump body with minimal seam or irregular flow surface. However, yet other embodiments contemplate graftable ducts that are assemblies of multiple components, such as by way of example only separate flexible ducts and less flexible housing connections joined by methods such as ultrasonic or other welding, as non-limited examples.

Each of the 4 grafts can then be connected to the pump body using a simple quick connect method (by way of example only, a flange clamp) to securely attach the rigid pre-formed ends of the grafts (i.e. flanges) to the pump. This novel method in some embodiments simplifies surgical implantation. Further, it can allow for pump exchange/upgrade to be performed more rapidly and with less health risk if pump change-out is ever necessary. It is further understood that although the use of ePTFE and PTFE is shown and described, various embodiments contemplate the use of any material.

This disclosure describes method and apparatus for surgical implantation of a blood pump. The pump uses in some embodiments 4 connections to blood vessels in a tight anatomic space. Using flexible PTFE grafts with rigid pre-formed connector ends (for connection to the pump housing) facilitates blood vessel sutured connections before the pump body is implanted. A ‘dummy’ (non-functional) pump sizer can be used by the surgeon to judge graft length and spacing. This dummy pump in some embodiments is a housing that preferably has the general form of the final implantable device. This dummy device can have only the external connections, although in other embodiments the dummy device can have an open interior chamber, such as a housing that is internally formed to support the final implantable pump or other components.

The grafts incorporate a flexible section of ePTFE that can be sutured to the patient anatomy. After the 4 grafts are properly placed, and vascular patency is assured, the pump can be put in position and connected to each of the 4 grafts preferably using a simple connector tool. This method also facilitates subsequent pump exchange, if ever needed, by allowing the surgeon to replace only the pump body without having to perform a delicate, complex, and risky dissection to perform new sutured connections to vascular structures that are located in critical spaces.

Flexible ePTFE grafts with rigid pre-formed PTFE connector ends can be used for suture connection to target blood vessels without having any obstruction from the body of the pump. The grafts can be tailored to the length and diameter needed, and can be sounded after suturing to ensure a widely patent connection. Once complete, the pump can be easily placed into position and attached using a simple clamp tool.

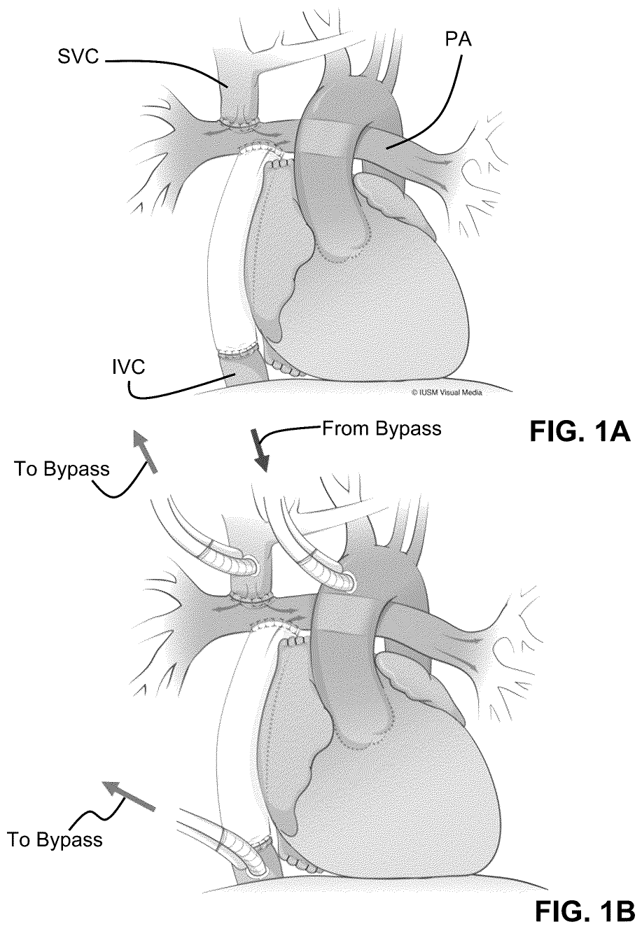

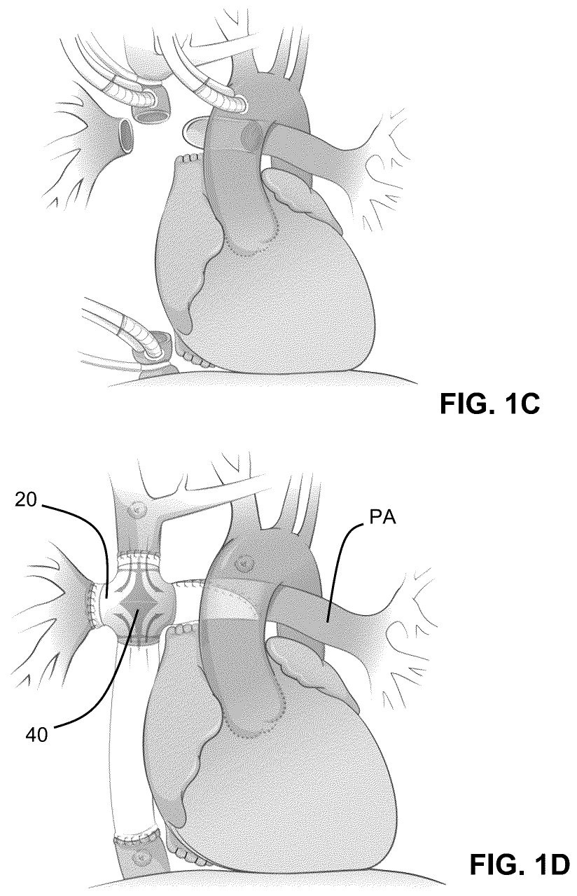

FIGS. 1A, 1B, and 1C each show a heart and the surrounding circulatory system being modified to include a Fontan junction. In FIG. 1D the junction is provided with an implantable pumping assembly 20 that is adapted and configured to provide a boost in blood pressure of less than about 15 mm Hg.

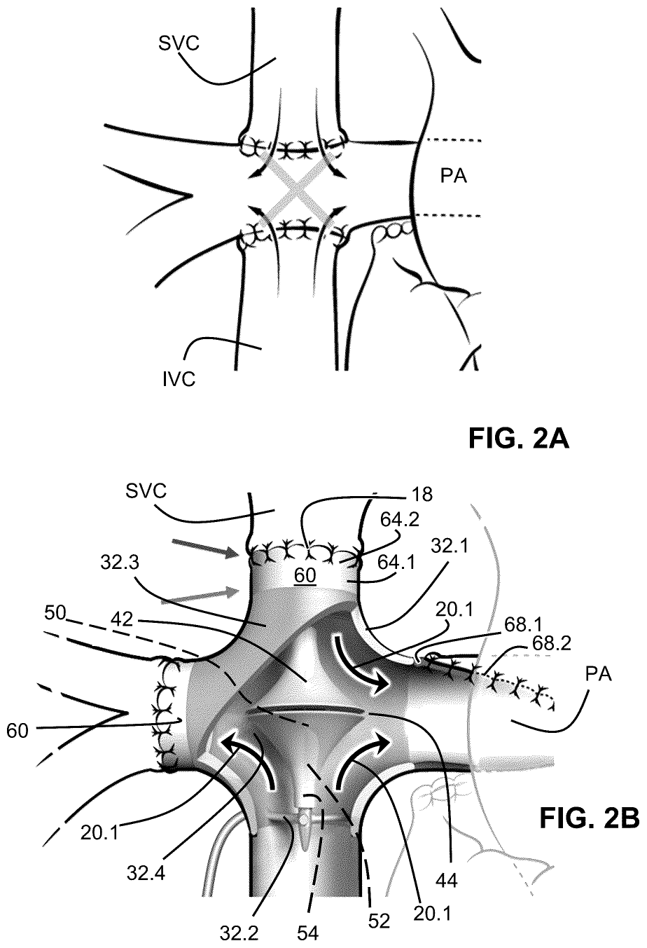

FIG. 2A is a close-up schematic representation of a Fontan junction, in which the superior vena cava (SVC) and the inferior vena cava (IVC) are modified to provide blood to the pulmonary artery (PA). FIG. 2B shows this same junction after an implantable pumping assembly 20 has been placed at the junction.

FIG. 2B shows a pumping assembly 20 in partial cross section. A rigid housing 20 supports a rotatable impeller 40 that is supported by bearings 54 and one or more struts 34.2. A motor 50 (not shown) is located within the impeller 40. The motor provides an electromagnetic field that spins the rotor. Blood flowing around the exterior of the rotor is viscously propelled by the rotor to exit the SVC or IVC and flow into the pulmonary artery. Also shown is an outlet 44 that provides an outlet for flow used to cool the motor 50.

FIG. 2B shows that the implantable pumping assembly 20 includes a housing 30 which in some embodiments (such as the one shown) includes a pair of opposing inlets 34 through which the rotor assembly is supported by struts 34.2. Blood is propelled to flow to a pair of outlets 38. Housing 30 comprises a body 32 having walls 32.1 that interconnect the inlets and outlets. The interior 32.2 of the housing is substantially open, and defines a chamber 32.4 in which the rotor is free to operate.

FIG. 2B further shows that each inlet 34 and each outlet 38 include a graftable conduit 60 that provides a connection interface between housing 30 and the associated vascular system. In one embodiment of the present invention, each conduit 60 comprises a flexible material such as ePTFE material. Each graftable conduit 60 includes a portion 64.1 (at the body inlets) and 68.1 (at the body outlets) that is adapted and configured to connect to the rigid or semi-rigid housing body 32. Each conduit 60 also includes, at the opposite end of the respective conduit, a portion 64.2 (inlet) or 68.2 (outlet) that is adapted and configured for connection to the circulatory system. As shown, each of the ends 64.2 and 68.2 are fabricated from a material (such as ePTFE) that is adapted and configured to be coupled to the circulatory system by sutures 18. The connections 64.1 and 68.1 can be made mechanically to body 32 in any manner.

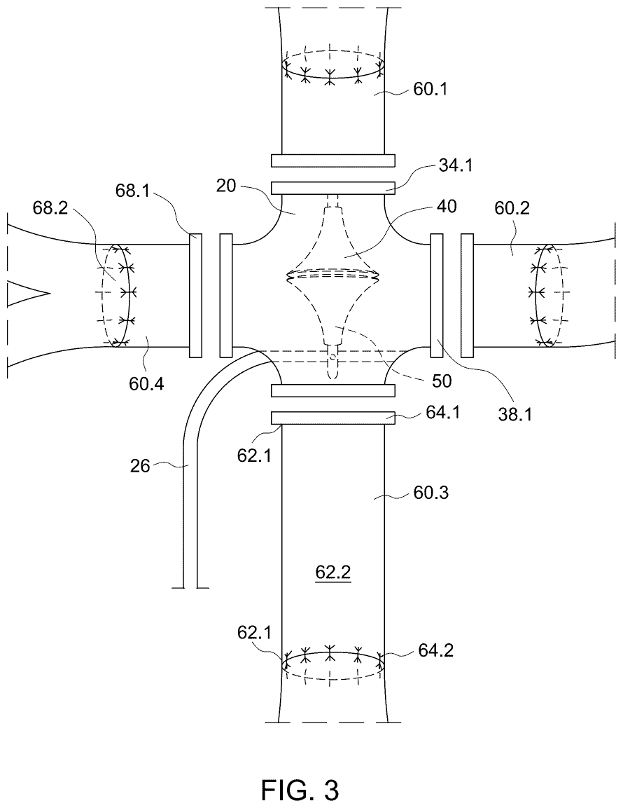

FIG. 3 is an exploded, perspective, line drawing of an installed implantable pumping assembly 20 according to another embodiment of the present invention. Placed centrally within FIG. 3 is an implantable pumping assembly 20 that operates substantially the same as the assembly 20 discussed with regards to FIG. 2B. It can be seen that a plurality of separable, graftable conduits 60 surround the pumping assembly 20, with a pair of conduits 60.1 and 60.3 arranged proximate to corresponding inlet connection features 34.1 of body 32. These inlet conduits 60.1 and 60.3 are shown at the top and bottom of FIG. 3. Also shown in FIG. 3 is a pair of separable, graftable conduits 60.2 and 60.4 shown on the right and left sides of body 32, and located proximate to the outlet connection features 38.1 of body 32. After the conduits are connected to the body, as will be described later, the lumen of each conduit is in fluid communication with the interior of body 32.

Various embodiments of the present invention contemplate that the graftable conduits 60 can be of different sizes in order to achieve proper flow conditions within the circulatory system. For example, the inner diameters of conduit 60.1 and 60.3 in some embodiments are 18 mm and 20 mm, respectively. The inner diameters of outlet conduits 60.2 and 60.4 are preferably 16 mm and 16 mm, respectively. It is further noted that each conduit can have a custom length from the housing connection end (64.1 or 68.1) to the anatomical connection end (64.2 or 68.2), as can be seen in comparing the schematic representations of inlet conduits 60.1 and 60.3.

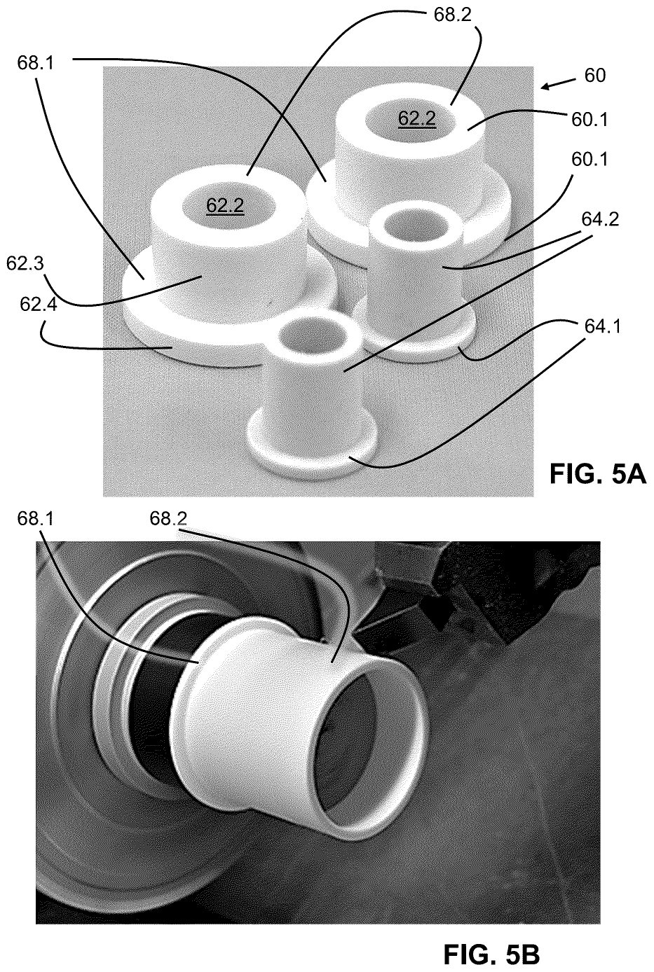

Further details of a graftable conduit 60 can be seen with reference to FIGS. 5A and 5B. FIG. 5A shows a plurality of conduit blanks. In some embodiments, each conduit 60 includes a body 62 having a pair of opposing ends 62.1 and defining a lumen 62.2 therebetween. Each conduit 60 preferably includes a flexible portion 62.3 and a rigid portion 62.4. FIG. 5B illustrates the conversion of one of the blanks 60 of FIG. 5A being converted into a graftable conduit 60 by stretching or otherwise modifying the PTFE into expanded PTFE (ePTFE).

In some embodiments, each conduit 60 is preferably a unitary construction without seams so as to eliminate potential failure sites. A conduit blank such as those shown in FIGS. 5A and 5B can be provided with a controlled temperature gradient with the use of appropriate heat sources and heat sinks, and fabricated to have a less rigid lumen section integrated with a more rigid flange section. As one example, the portion of the blank to be formed into flexible portion 62.3 can be exposed to a localized source of heat. The portion of the blank to be formed into rigid portion 62.4 can be exposed to a localized source of active cooling, or to a heat sink. The blank can then be formed such as by drawing or other means to stretch and thin the lumen section 62.3 and preferably expand the native PTFE. It may be helpful during final curing to control the rate of cooling after the conduit has been initially formed, especially for purposes of maintaining appropriate density and shortening. In some embodiments, the flange portion 62.4 remains with the native material properties.

Still further, the stretch ratio applied to the blank can be varied along the axial length of the blank, with the wall of the flexible portion preferably being thinner than the wall that is proximate to the rigid (flanged) section so as to result in an acceptable stress profile in the geometry transition from the cylindrical wall of the conduit to the thicker flange.

Preferably, the finished conduit will have a flexible walled section that is softer, more compressible, more flexible, and accommodate greater tissue growth (endothelization) than the flanged portion. Likewise, the rigid, flanged portion of the conduit will be stiffer, less compressible, less flexible, and accommodate less endothelization than the flexible portion.

Although what is shown and described are conduits 60 that comprise PTFE and ePTFE, various embodiments of the present invention contemplate conduits 60 that can be fabricated from any kind of biocompatible material. Preferably, each conduit 60 is of a unitary, integrated structure comprising a rigid or semi rigid housing connection end 68.1 (for an outlet) that is integral with a flexible, suturable anatomical connection end 68.2. In one embodiment, a graftable conduit is fabricated from a PTFE blank that is rigid or semi-rigid.

The rigid end 68.1 is preferably modified to include a flange at the housing connection end. Moving distally from that housing connection end, the cylindrical selection of the blank is then appropriately modified to ePTFE, which results in the cylindrical section being flexible as compared to the flange end. With this flexibility, along with the reduced wall thickness of the anatomical connection end, the anatomical connection end becomes adapted and configured for connection by suture to the circulatory system.

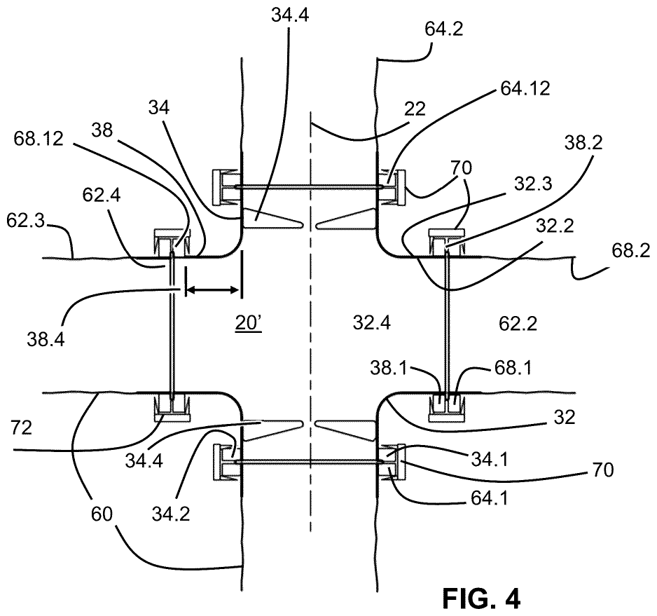

FIG. 4 shows a cross sectional schematic representation of a mock-up or dummy implantable assembly 20′ that is coupled to a plurality of conduits. As shown in FIG. 4, a body 32 (not including an impeller 40 or motor 50) is shown interconnected to a plurality of conduits (as shown exploded from the body in FIG. 3). Some embodiments of the present invention include the use of an empty housing body as a means to perform a fit check and verify the proper placement of the graftable conduits 60. After the correct placement has been demonstrated, the dummy pumping assembly 20′ can be easily removed and replaced with the functioning pumping assembly 20, in a manner as will be described.

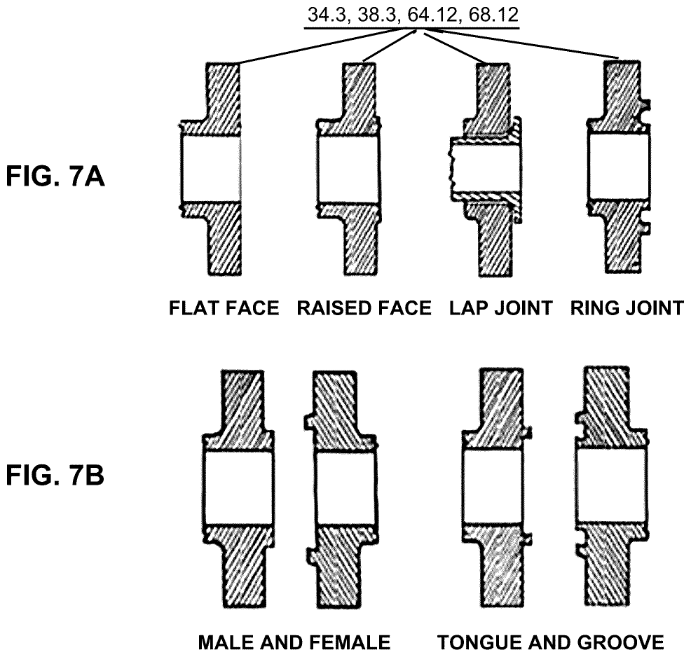

FIG. 4 shows that the housing connection ends 64.1 (inlet) or 68.1 (outlet) each preferably include a preferably flat, outwardly extending face or flange that is placed in abutting relationship with the substantially flat face of the respective housing connection feature 34.1 (inlet) or 38.1 (outlet). Although what has been shown and described is flange to flange interface that is substantially flat, further embodiments of the present invention contemplate the pairing of any structure of conduit to body interfaces other than flanges, and any manner of interface between the faces of the housing and conduit interfacing structures, including structures adapted and configured to provide locating features or sealing features. Various types and configurations of flange locating features are shown in FIGS. 7A and 7B. Further, it is understood that the flange to flange mating interface can include accommodations (such as a groove) for a seal.

Referring again to FIG. 4, after the housing flange and the conduit flange are placed in abutting relationship to each other, a clamp 70 is placed to substantially surround the outer diameter of the pair of flanges. Preferably, this clamp 70 extends substantially around the entirety of the periphery of the mating flanges. In some embodiments, the clamp 70 includes a channel 72 that is adapted and configured to provide compression of the flanges against each other. In the embodiment shown, channel 72 includes one or more angled internal surfaces that provide the face to face compression as the clamp 70 is tightened around the periphery of the connect flanges. In this embodiment, a wedging action occurs as the open and loosened clamp 70 is tightened into place. A non-limiting example of a clamp useful in some embodiments is similar to a Marman clamp. FIG. 4 further show an example of a gap 38.4 between a housing flange and an external surface of the housing. This gap permits sufficient clearance to prevent interference between the clamp and the housing, and in some embodiments also provides sufficient clearance to prevent interference between adjacent clamps.

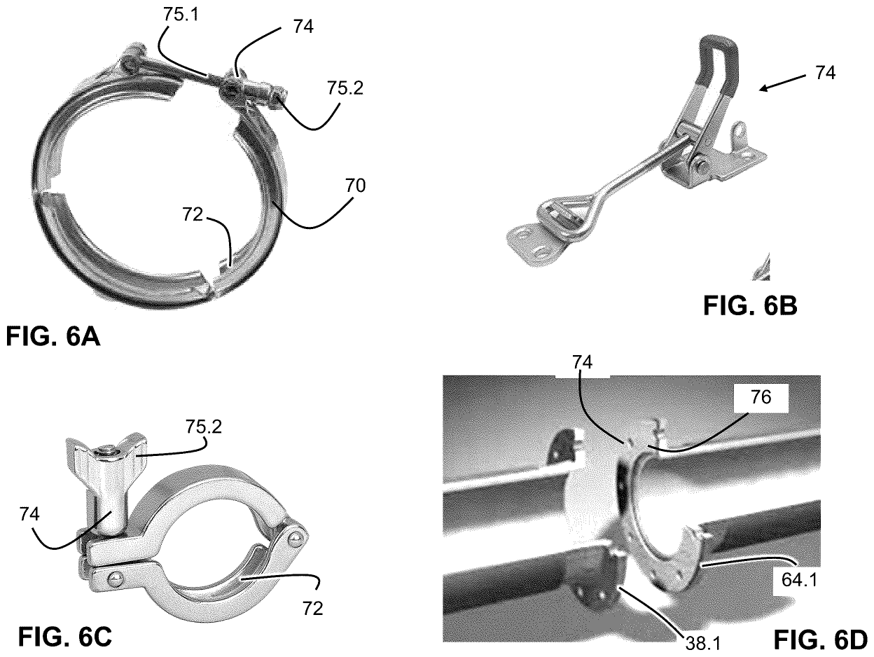

FIG. 6A shows an example of one such clamp. Clamp 70 of FIG. 6A includes a channel 72 provided on a multi-section split ring, with the clamp including a locking mechanism 74 located at the split. It can be seen that at the split there is a pivotal bolt having a threaded section 75.1 that extends through an eye on the opposite side of the split. As the nut 75.2 is tightened on the threaded portion, the extended, opened diameter of the lock is reduced to a locking configuration, from which the channel 72 applies compression to the mating flange faces. FIG. 6C shows yet a different clamping assembly 70 that can be used to apply compression to the flange faces. FIG. 6B shows yet a further device for pulling together the split ends of a clamp. It is understood that each end of the lock 74 of FIG. 6B is shown by itself, without the channeled split ring. Still further, FIG. 6D shows another manner of providing compression between two flange faces by way of a plurality of nuts and bolts (not shown) or other fasteners that extend between mating holes in the flanges. In some embodiments, a seal 76 such as a gasket or O-ring is further provided between the flange faces.

FIGS. 7A and 7B show a variety of flange configurations useful in various embodiments of the present invention. It is understood that each of the flange configurations shown in these two figures represent cross sectional depictions through the duct centerline of flanges having preferably circular peripheries.

The flange identified as “flat face” is the same as the flange faces depicted in FIG. 4. These faces are substantially flat and smooth, and preferably have faces that are generally orthogonal to either the duct flange periphery or the duct centerline. These faces can be brought together in a sealing configuration with any of the means for compressing flange faces shown in FIG. 3, 4, or 6. It is further understood that the means for compression shown in FIG. 6D does not include a ring surrounding the periphery of the flange, but rather depicts flange faces in which at least one flange contains a through hole that aligns with a through hole (or a threaded hole) in the mating aperture, thus comprising a bolted flange assembly.

FIG. 7A shows other cross sectional configurations of flanged joints, including the three labeled raised face, lap joint, and ring joint. It is understood that any of the four cross sections shown in FIG. 7A can be used for any of the outlet duct, inlet duct, housing outlet, or housing inlet as shown and described herein. It is understood that use of the raised face, lap joint, or ring joint in various embodiments may be facilitated by having additional features useful with the particular joint (for example, having a sealing ring to fit within the ring groove of the ring joint).

FIG. 7B shows two pairs of flange configurations, male and female. In the two figures labeled male and female, it can be seen that one flange face would include a male feature that is received within the female feature of the opposing flanged joint. Likewise, in the pair of figures labels tongue and groove it can be seen that this same idea is present in one face having a male projection that fits within a female groove on the other face. The combinations of flange faces shown in FIG. 7B include a further coupling feature which is means for aligning the duct with the housing. It is also understood that the various clamps shown in FIG. 6 include yet other features for alignment of the two faces. In FIGS. 6A, 6B, and 6C, the alignment is achieved when the periphery of the flanges is captures within the channels, the channels preferably being adapted and configured to have the inner wall of the channel pressed against the periphery of the adjoined clamps. FIG. 6D shows the use of bolts to provide alignment.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

What is claimed is:

1. An apparatus for implantation in the circulatory system of an animal, comprising:
a housing comprised of a biocompatible material and including an interior and a chamber within the interior, the chamber being adapted and configured for internal support of a rotor, the housing having first, second, and third duct connection features each adapted and configured for connection to a fluid duct;
a first separable graftable duct having an anatomical connection end and a housing connection end and including therebetween a lumen adapted and configured for the flow of blood;
a second separable graftable duct having an anatomical connection end and a housing connection end and including therebetween a lumen adapted and configured for the flow of blood; and
a third separable graftable duct having an anatomical connection end and a housing connection end and including therebetween a lumen adapted and configured for the flow of blood;
wherein each anatomical connection end comprises a material adapted and configured for connection by sutures to a circulatory system, and the first, second, and third duct connection features are each adapted and configured for connection to the corresponding first, second, and third housing connection ends.

2. The apparatus of claim 1 wherein the housing connection end of each graftable duct includes polytetrafluorethylene.

3. The apparatus of claim 2 wherein the anatomical connection end of each graftable duct includes expanded polytetrafluorethylene.

4. The apparatus of claim 3 wherein each said first duct, said second duct, and said third duct are seamless unitary ducts.

5. The apparatus of claim 1 wherein each said first, second, and third duct connection features include a flange being spaced apart from the exterior surface of said housing by a gap.

6. The apparatus of claim 1 wherein each said first, second, and third housing connection ends include a flange adapted and configured for repeatedly releasable connection to the corresponding first duct connection feature, second duct connection feature, or third duct connection feature.

7. An apparatus for implantation in the circulatory system of an animal, comprising:
a housing comprised of a biocompatible material and including an interior, the housing having first and second fluid inlets and at least one fluid outlet with each of the first and second fluid inlets and the fluid outlet being in fluid communication with the interior;
a first separable graftable inlet duct comprised of a biocompatible material and having two ends and including first means for coupling at one end to a circulatory system and first means for coupling at the other end to the housing first fluid inlet and including between the two ends of said first inlet duct a wall surrounding a lumen adapted and configured for the flow of the fluid;
a second separable graftable inlet duct comprised of a biocompatible material and having two ends and including second means for coupling at one end to the circulatory system and second means for coupling at the other end to the housing second fluid inlet and including between the two ends of said second inlet duct a wall surrounding a lumen adapted and configured for the flow of the fluid; and
a third separable graftable outlet duct including comprised of a biocompatible material and having two ends and outlet duct means for coupling at one end to the circulatory system and outlet duct means for coupling at the other end to the housing outlet and including between the two ends of said outlet duct a lumen adapted and configured for the flow of the fluid.

8. The apparatus of claim 7 wherein at least one of said first means for coupling to a circulatory system, said second means for coupling to a circulatory system, and said third means for coupling to a circulatory system is by using sutures to couple said one means to the circulatory system.

9. The apparatus of claim 7 wherein at least one of said first means for coupling at the other end to the first inlet, said second means for coupling at the other end to the second inlet, and said third means for coupling at the other end to the outlet includes a circular flange.

10. The apparatus of claim 7 wherein said first means for coupling at the other end includes a first duct circular flange having a flat face, said second means for coupling at the other end includes a second duct circular flange having a flat face, and said outlet duct means for coupling at the other end includes a third duct circular flange having a flat face.

11. The apparatus of claim 7 wherein said housing first fluid inlet includes a first housing flange for coupling said first duct to the first fluid inlet, said housing second fluid inlet includes a second housing flange for coupling said second duct to the second fluid inlet, and said housing fluid outlet includes a third housing flange for coupling said third duct to the fluid outlet,

12. The apparatus of claim 11 wherein said first means for coupling at the other end includes a first duct flange, said second means for coupling at the other end includes a second duct flange, and said outlet duct means for coupling at the other end includes a third duct flange.

13. The apparatus of claim 11 which further comprises first means for compressing said first flange to said first duct, second means for compressing said second flange to said second duct, and third means for compressing said third flange to said third outlet duct.

14. The apparatus of claim 13 wherein said first means for compressing includes a first circular clamp that surrounds the periphery of the first flange, said second means for compressing includes a second circular clamp that surrounds the periphery of the second flange, and said third means for compressing includes a third circular clamp that surrounds the periphery of the third flange.

15. The apparatus of claim 13 wherein said first means for compressing, said second means for compressing, and said third means for compressing each include fasteners.

16. The apparatus of claim 11 wherein each said first flange, said second flange, and said third flange includes a circular periphery and an outwardly facing flat surface.

17. The apparatus of claim 7 wherein the fluid is blood.

18. The apparatus of claim 18 wherein said means for coupling at one end to the circulatory system of the at least one of said means for coupling at the other end is by using sutures.

19. The apparatus of claim 7 wherein each of said first means for coupling at one end to a circulatory system, said second means for coupling at one end to a circulatory system, and said outlet duct means for coupling at one end to the circulatory system comprise a material that is adapted and configured for suturing.

20. The apparatus of claim 7 wherein each said means for coupling at one end to a circulatory system is substantially flexible, and each said means for coupling at the other end to the housing is substantially rigid.

21. The apparatus of claim 7 wherein each said means for coupling at one end to a circulatory system comprises polytetrafluorethylene that is expanded, and each said means for coupling at the other end to the housing comprises polytetrafluorethylene.

22. The apparatus of claim 7 wherein each said first duct, said second duct, and said third duct are seamless and each are a unitary structure.

23. The apparatus of claim 7 wherein said housing includes at least a first strut located within the interior and proximate to the first inlet and at least a second strut located within the interior and proximate to the second inlet, said first and second strut each being adapted and configured to support a shaft extending therebetween and through the chamber.

24. An apparatus for implantation in the circulatory system of an animal, comprising:
an implantable housing comprised of a biocompatible material and having at least three sides and including an interior and a chamber within the interior, the housing having at least three fluid connection ports with each of the fluid connection ports being in fluid communication with the chamber, two of the fluid connection ports being located on opposite respective first and second sides of the housing, the fluid connection port being located on a third side of the housing in between the first and second sides;
wherein each fluid connection port includes a flange located on the exterior of said housing and extending circumferentially around the respective fluid connection port, each flange being spaced apart from the exterior surface of said housing by a gap.

25. The apparatus of claim 24 wherein the interior of said housing is adapted and configured to include a rotating pumping element.

26. The apparatus of claim 24 wherein each flange has a circular circumference and a substantially flat outwardly facing surface.

27. An apparatus for implantation in the circulatory system of an animal, comprising:
a duct fabricated from a biocompatible material and adapted and configured for implantation into an animal, said duct including a substantially flexible lumen section with two opened ends, with the first end being opened end adapted and configured for suturing connection to the circulatory system, said duct including a substantially rigid flange located at the second end, the flange including an opened aperture in fluid communication with the second end of the lumen, the lumen section and the flange comprising a unitary seamless structure.

28. The apparatus of claim 1 wherein the housing has an external surface, and at least one of the first, second, and third duct connection features includes a flange having a housing-facing inner face separated from the external surface by a gap and a duct-facing outer face, the inner face having a periphery adapted and configured to be compressed against a corresponding one of said first, second, and third housing connection ends with a clamp.

29. The apparatus of claim 1 wherein at least one of the first, second, and third duct connection features includes a duct-facing outer face, the corresponding one of said first, second, and third housing connection ends having a housing facing surface, and each of the duct-facing outer face and housing facing surface include means for alignment.
